(19) United States
(12) Patent Application Publication
(10) Pub. No.: US 2026/0053773 A1
(43) Pub. Date: Feb. 26, 2026

(54) PHARMACEUTICAL COMPOSITION AND USE THEREOF

(71) Applicant: AUHUI TONGOO PHARMACEUTICAL TECHNOLOGY CO., LTD., Hefei, Anhui (CN)

(72) Inventors: Xianyuan CHENG, Hefei, Anhui (CN); Weiwei TANG, Hefei, Anhui (CN); Si LI, Hefei, Anhui (CN); Junbo GONG, Hefei, Anhui (CN); Ying FENG, Hefei, Anhui (CN)

(21) Appl. No.: 19/102,416

(22) PCT Filed: Aug. 30, 2023
(86) PCT No.: PCT/CN2023/115656

§ 371 Date: Feb. 10, 2025

(30) Foreign Application Priority Data
Aug. 30, 2022 (CN) 202211043899.6

(51) Int. Cl. A61K 31/366; A61K 31/05; A61K 31/192; A61K 31/194; A61K 31/353; A61K 31/7034; A61K 31/7048

(52) U.S. Cl. CPC A61K 31/366; A61K 31/05; A61K 31/192; A61K 31/194; A61K 31/353; A61K 31/7034; A61K 31/7048

(57) ABSTRACT

Provided are a pharmaceutical composition and use thereof. The pharmaceutical composition includes a component A and a component B. The component A includes polyphenol having 6 to 14 carbon atoms and/or a substance that is metabolizable into polyphenol having 6 to 14 carbon atoms in the human body. The component B includes any one of citric acid, citrate, a citric acid derivative, or a citric acid derivative salt, or a combination of at least two thereof. The pharmaceutical composition not only has a synergistic enhancement effect in inhibiting the growth of calcium oxalate crystals, but also can be used for a synergistic treatment of kidney stones.

### PRIORITY INFORMATION

This application claims priority and benefits of the patent application No. 202211043899.6 filed with the China National Intellectual Property Administration on Aug. 30, 2022, which is incorporated herein by reference in its entirety.

### FIELD

The present disclosure belongs to the field of biomineralization and biomedicine, and specifically relates to a pharmaceutical composition and a use thereof, and more particularly, to a combined pharmaceutical composition for treating kidney stones and a use thereof.

### BACKGROUND

Biomineralization is a common phenomenon in nature, and it is divided into normal mineralization (such as teeth, shells, eggshells, etc.) and pathological mineralization (kidney stones, gallstones, gout, etc.). Because the pathological mineralization process mainly involves a series of crystallization processes, such as the accumulation of supersaturation of solute molecules, the nucleation of solute, crystal growth, and crystal aggregation, etc. for the treatment of most diseases related to pathological crystallization, currently the treatment and prevention of related diseases have been achieved from a perspective of crystallization by inhibiting a pathological crystallization process.

The kidney stones are a common disease in the urinary system. The incidence of kidney stones in China in 2016 was 7.54%, and recurrence rate of stones within 5 to 10 years was as high as 50%. Therefore, the treatment and prevention of stones have received widespread attention in clinical practice. Because calcium oxalate is the main component of kidney stones, current treatments for kidney stones from the perspective of crystallization mainly focus on inhibiting the crystallization of calcium oxalate stones. Studies have found that calcium oxalate monohydrate (COM) and calcium oxalate dihydrate (COD) exist in the bodies of normal people and patients with stones. COM crystals are easy to adhere to renal epithelial cells and macromolecules in urine, grow and aggregate to form large particles, which eventually cannot be excreted and remain in the kidneys to form stones, so COM is the main component of kidney stones. Therefore, from the perspective of crystallization, inhibiting the growth of COM crystals can effectively prevent the formation of stones and reduce the recurrence rate of stones.

Related studies report that COM crystal growth inhibitors include metal ions, small organic molecules, and macromolecules. Among them, Na⁺, K⁺, Li⁺, and Cs⁺ can not only reduce the supersaturation of calcium oxalate, but also inhibit macroscopic growth of the COM crystals, with an inhibition rate of 50%; citric acid has an inhibitory effect of 60% on calcium oxalate crystallization; polyphosphate substances can reduce appearance of COM by inducing the production of COD; and urine proteins, such as osteopontin, chondroitin sulfate, etc. have an inhibitory effect of only 30% to 40% on calcium oxalate crystallization. Therefore, a class of COM crystal growth inhibitors is needed to completely inhibit the growth of the COM crystals and reduce the formation of the stones, thereby preventing the stones.

In recent years, combined administration of drugs has gradually become a research hotspot in the treatment of diseases. The combined administration of drugs can exert a synergistic effect of drugs and achieve a better disease treatment effect. However, when drugs are used in combination, the drug interactions may also produce opposite results. Therefore, in this field, efforts have been made to develop inhibitors for calcium oxalate crystallization and to search for drug combinations with synergistic effect to further enhance the prevention effect of stones.

### SUMMARY

On the basis of the existing technology, in order to further enhance the effect of treatment and prevention of stones, the present disclosure provides a pharmaceutical composition and use thereof to improve the therapeutic effect of a drug by means of synergistic enhancement.

One of the objects of the present disclosure is to provide a pharmaceutical composition. The pharmaceutical composition includes a component A and a component B. The component A includes polyphenol having 6 to 14 carbon atoms and/or a substance that is metabolizable into polyphenol having 6 to 14 (for example 6, 10, 12, 14, and the like) carbon atoms in human body. The component B includes any one of citric acid, citrate, a citric acid derivative, or a citric acid derivative salt, or a combination of at least two thereof.

According to an embodiment of the present disclosure, the polyphenol having 6 to 14 carbon atoms includes any one of ellagic acid, gallic acid, pyrogallol, or protocatechuic acid, or a combination of at least two thereof.

According to an embodiment of the present disclosure, the substance that is metabolizable into a polyphenol having 6 to 14 carbon atoms in the human body includes any one of tannic acid, punicalagin, galloyl glucose, or esterified catechin, or a combination of at least two thereof.

According to an embodiment of the present disclosure, the citrate includes any one of potassium citrate, sodium citrate, or magnesium citrate, or a combination of at least two thereof.

According to an embodiment of the present disclosure, the citric acid derivative includes hydroxycitric acid, an ester compound derived from citric acid, or a lactone compound derived from citric acid.

According to an embodiment of the present disclosure, the citric acid derivative salt includes any one of potassium hydroxycitrate and/or sodium hydroxycitrate, or magnesium hydroxycitrate, or a combination of at least two thereof.

According to an embodiment of the present disclosure, a molar ratio of the component A to the component B in the pharmaceutical composition is (0.0013 to 3.33): 1.

According to an embodiment of the present disclosure, a combined effecting concentration ratio of the component A and the component B is set based on effect mechanisms of the respective components, exemplarily including:
when the effect mechanisms of the component A and the component B are the same, a molar ratio of the component A to the component B being (0.0013 to 0.163): 1, for example, 0.005:1, 0.008:1, 0.01:1, 0.03:1, 0.05:1, 0.08:1, 0.1:1, 0.12:1, 0.15:1, 0.16:1, and the like;
when the effect mechanisms of the component A and the component B are similar, the molar ratio of the component A to the component B being (0.0023 to 0.025): 1, for example, 0.003:1, 0.005:1, 0.008:1, 0.01:1, 0.012:1, 0.015:1, 0.018:1, 0.02:1, 0.022:1, 0.025:1, and the like; or
when the effect mechanisms of the component A and the component B are different, the molar ratio of the component A to the component B being (1.77 to 3.33): 1, for example, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, and the like.

According to an embodiment of the present disclosure, the pharmaceutical composition includes ellagic acid with a molar concentration ranging from 0 μM to 2 μM (e.g., 0.1 μM, 0.3 μM, 0.5 μM, 0.7 μM, 1 μM, 1.2 μM, 1.5 M, 1.8 μM, etc.) and a component B with a molar concentration ranging from 0 mM to 0.5 mM (e.g., 0.1 mM, 0.2 mM, 0.3 mM, 0.4 mM, etc.); and an addition amount of the ellagic acid and an addition amount of the component B are not zero. Preferably, the pharmaceutical composition includes ellagic acid and citric acid or potassium citrate or hydroxycitric acid.

According to an embodiment of the present disclosure, the pharmaceutical composition includes gallic acid with a molar concentration ranging from 0 μM to 25 μM (e.g., 1 μM, 3 μM, 5 μM, 7 μM, 10 μM, 12 μM, 15 μM, 18 μM, 20 μM, 22 μM, etc.) and a component B with a molar concentration ranging from 0 mM to 0.5 mM (e.g., 0.1 mM, 0.2 mM, 0.3 mM, 0.4 mM, etc.); and an addition amount of the gallic acid and an addition amount of the component B are not zero. Preferably, the pharmaceutical composition includes gallic acid and citric acid or potassium citrate or hydroxycitric acid.

According to an embodiment of the present disclosure, the pharmaceutical composition includes pyrogallol with a molar concentration ranging from 0 mM to 15 mM (e.g., 1 mM, 3 mM, 5 mM, 7 mM, 10 mM, 12 mM, 15 mM, etc.) and a component B with a molar concentration ranging from 0 mM to 0.5 mM (e.g., 0.1 mM, 0.2 mM, 0.3 mM, 0.4 mM, etc.); and an addition amount of the pyrogallol and an addition amount of the component B are not zero. Preferably, the pharmaceutical composition includes pyrogallol and citric acid.

According to an embodiment of the present disclosure, the pharmaceutical composition includes protocatechuic acid with a molar concentration ranging from 0 mM to 0.55 mM (e.g., 0.05 mM, 0.1 mM, 0.15 mM, 0.2 mM, 0.25 mM, 0.3 mM, 0.35 mM, 0.4 mM, 0.45 mM, 0.5 mM, etc.) and a component B with a molar concentration ranging from 0 mM to 0.5 mM (e.g., 0.1 mM, 0.2 mM, 0.3 mM, 0.4 mM, etc.); and an addition amount of the protocatechuic acid and an addition amount of the component B are not zero. Preferably, the pharmaceutical composition includes protocatechuic acid and citric acid.

According to an embodiment of the present disclosure, the pharmaceutical composition further includes a pharmaceutically acceptable carrier or excipient.

According to an embodiment of the present disclosure, the carrier includes any one of liposomes, micelles, dendritic macromolecules, microspheres, or microcapsules, or a combination of at least two thereof. The present disclosure does not limit specific materials of the carrier, which can be limited by those skilled in the art according to the actual needs.

According to an embodiment of the present disclosure, the excipient includes any one of a wetting agent, a binder, or a disintegrant, or a combination of at least two thereof. The present disclosure does not limit specific materials of the excipient, which can be limited by those skilled in the art according to the actual needs.

According to an embodiment of the present disclosure, the pharmaceutical composition is in a formulation of compound preparation.

According to an embodiment of the present disclosure, the pharmaceutical composition is in a formulation of a separate preparation of the component A and a separate preparation of the component B.

According to an embodiment of the present disclosure, an administration of the separate preparation includes simultaneous administration, separate administration, or sequential administration.

According to an embodiment of the present disclosure, a dosage form of the pharmaceutical composition includes any pharmaceutically acceptable dosage form.

According to an embodiment of the present disclosure, the dosage form includes tablets, granules, powders for solution, pulveres, capsules, injections, and the like.

A second object of the present disclosure is to provide a use of the pharmaceutical composition according to the first object in the preparation of a medicament for preventing and/or treating kidney stones.

A third object of the present disclosure is to provide a use of the pharmaceutical composition according to the first object in the preparation of a crystal growth inhibitor for calcium oxalate monohydrate.

A fourth object of the present disclosure is to provide a use of the pharmaceutical composition according to the first object in the prevention and/or treatment of kidney stones.

A fifth object of the present disclosure is to provide a method for preventing and/or treating kidney stones, including administering an effective amount of the pharmaceutical composition according to the first object to a subject suffering from or suspected of suffering from kidney stones.

Compared with the prior art, the present disclosure has the following beneficial effects.

The present disclosure verifies through experiments that the combination of the component A and the component B can inhibit the growth of COM crystals, and the combination of the two components exhibits a synergistic effect compared with the use of either component A or component B alone. In particular, the combination of ellagic acid, gallic acid, pyrogallol, or protocatechuic acid with citric acid can inhibit the growth of COM crystals, and the combination of the two components exhibits a better synergistic effect compared with the use of ellagic acid, gallic acid, pyrogallol, protocatechuic acid, or citric acid alone. Furthermore, when the ellagic acid, gallic acid, pyrogallol, or protocatechuic acid is used in combination with citric acid, even if the respective doses are halved, the inhibition effect on the growth of COM crystal is stronger than any one of them was used alone

The present disclosure verifies that ellagic acid, pyrogallol, and citric acid share a same mechanism in inhibiting the growth of COM crystals through experiments, which is a combination mechanism of step pinning and kink blocking, and the two inhibitor molecules can play a synergistic effect. Specifically, on the other hand, the inhibitor molecules, such as ellagic acid, pyrogallol, or citric acid, can be adsorbed on the crystal terrace of COM crystal face, hindering the extension of growing ledge. When an extending ledge encounters the inhibitor molecules adsorbed on the terrace, the edge of the ledge bends, and the bent ledge has a higher solubility, thereby the local supersaturation of the ledge was reduced and thus reducing the growth rate of the ledge. When the distance between inhibitor molecules adsorbed on the terrace is less than the critical diameter for ledge growth corresponding to the supersaturation of the solution, the ledge growth stops, and the crystal face is completely inhibited. On the other hand, these inhibitors can be adsorbed onto the kinks on the crystal ledge, to reduce the density of growth nodes used for solute embedding, and decrease the ledge growth kinetic coefficient, and thus reducing the ledge growth rate. When ellagic acid or pyrogallol is used in combination with citric acid, and the distance between different inhibitor molecules adsorbed on the terrace is smaller than the distance between the same inhibitor molecules, the combination of the two inhibitors exhibits a stronger inhibition effect.

The present disclosure verifies that gallic acid and citric acid have similar mechanisms in inhibiting COM crystal growth through experiments, wherein the mechanism of gallic acid is only step pinning, but the combination of two inhibitor molecules exerts a synergistic effect. The specific mechanism of gallic acid is that the gallic acid molecules are adsorbed onto the terrace of COM crystal face, which reduces the local supersaturation of the growing ledge, and thus reducing the growth rate of the ledge until crystal face growth is completely inhibited. When the gallic acid and citric acid are used in combination, the distance between different inhibitor molecules adsorbed on the terrace is shorter than the distance between the same inhibitor molecules, the combination of the two inhibitors exhibits a stronger inhibition effect.

The present disclosure verifies that although protocatechuic acid and citric acid have different mechanisms in inhibiting the growth of COM crystal through experiments, wherein the mechanism of protocatechuic acid is kink blocking, the combination of the two inhibitor molecules plays a synergistic effect. The specific mechanism of protocatechuic acid is that the protocatechuic acid molecules are adsorbed on the growth kinks on the terrace of COM crystal face, to reduce the density of growth nodes used for solute embedding and decrease the ledge growth kinetic coefficient, and thus reducing the ledge growth. The protocatechuic acid molecules and citric acid molecules are adsorbed on the growth kinks and terrace, respectively, and these two processes do not conflict with each other. When the distance between the two is smaller than the distance between the citric acid molecules, the combination of the two inhibitors can achieve a synergistic effect.

The combined pharmaceutical composition of ellagic acid, gallic acid, pyrogallol, or protocatechuic acid with citric acid according to the present disclosure can improve the inhibition effect of a single inhibitor molecule on COM crystals, reduce the formation and growth of COM crystals, thereby improving the preventive effect of the inhibitor drug on kidney stones.

Additional aspects and advantages of the present disclosure will be provided in part in the following description, or will become apparent in part from the following description, or can be learned from practicing of the present disclosure.

### BRIEF DESCRIPTION OF THE DRAWINGS

The above and/or additional aspects and advantages of the present disclosure will become apparent and easily understood from the description of the embodiments in conjunction with the following figures, in which:

FIG. 1 is an isobologram of ellagic acid and citric acid when used in combination in the inhibition of COM crystal growth;

FIG. 2 is an isobologram of gallic acid and citric acid when used in combination in the inhibition of COM crystal growth;

FIG. 3 is an isobologram of pyrogallol and citric acid when used in combination in the inhibition of COM crystal growth;

FIG. 4 is an isobologram of protocatechuic acid and citric acid when used in combination in the inhibition of COM crystal growth;

FIG. 5 is a colored image of human proximal tubule cells, survival (green) and death (red), with a scale bar of 50 μm;

FIG. 6 is a statistical analysis of cell death/survival ratio. Compared with the control group, *p<0.05, ** p<0.01; and compared with the CA group, #p<0.05, ##p<0.01;

FIG. 7 shows percentage of lactate dehydrogenase release. Compared with the control group, *p<0.05, ** p<0.01; and compared with the CA group, #p<0.05, ##p<0.01; and

FIG. 8 shows number of COM crystal adhesions. Compared with the control group, *p<0.05, ** p<0.01; and compared with the CA group, #p<0.05, ##p<0.01.

### DETAILED DESCRIPTION

The technical solution of the present disclosure is further illustrated below through specific embodiments. It should be clear to those skilled in the art that the embodiments are only intended to help understand the present disclosure and should not be considered as specific limitations of the present disclosure.

It should be noted that the terms such as “first” and “second” are used herein for purposes of description only and should not be understood as to indicating or implying relative importance, or to implicitly indicating the number of technical features indicated. Thus, the features defined with “first” and “second” may explicitly or implicitly include one or more such features. Further, in the description of the present disclosure, “a plurality of” means two or more than two, unless specified otherwise.

The endpoints and any value of the ranges disclosed herein shall not limited to the exact range or value, and those ranges or values should be understood to include values close to those ranges or values. For numerical ranges, the endpoints of each range, the endpoints of each range and the individual point values, and the individual point values may be combined with each other to obtain one or more new numerical ranges, which should be deemed to be specifically disclosed herein.

In order to understand the present disclosure more readily, certain technical and scientific terms are specifically defined below. Unless explicitly defined otherwise in this document, all other technical and scientific terms used herein have the same meanings as commonly understood by those skilled in the art to which the present disclosure belongs.

As used herein, the terms “include” or “including” are open expressions, that is, including the contents specified in the present disclosure but not excluding other contents.

As used herein, the terms “optionally”, “optional”, or “optionally” generally mean that the event or condition subsequently described may, but need not, occur, and the description includes situations in which the event or condition occurs, as well as situations in which the event or condition does not occur.

As used herein, the term “composition” exists in a form that permits the biological activity of the active ingredients to be effective, and does not contain additional ingredients that are unacceptably toxic to a subject to whom the composition would be administered.

As used herein, the term “treatment” refers to the elimination, reduction, suppression, or amelioration, temporarily or permanently, partially or completely, of the clinical symptoms, manifestations, or progression of an event, disease, or symptom.

As used herein, the terms “subject” and “patient” can be used interchangeably, regardless of whether the subject has received or is currently receiving any form of treatment. As used herein, the term “subject” or “patient” refers to a mammalian subject or patient. Unless otherwise indicated, the terms “patient” or “subject” are used interchangeably herein. Exemplary subjects include, but are not limited to, humans, monkeys, dogs, cats, mice, rats, cows, horses, camels, birds, goats, and sheep. In some embodiments, the subject is a human. In some embodiments, the subject is a human suspected of having cancer, an autoimmune disease or condition, and/or an infection.

The solutions of the present disclosure will be explained below in conjunction with examples. Those skilled in the art will appreciate that the following examples are only used to illustrate the present disclosure and should not be considered as limiting the scope of the present disclosure. Where specific techniques or conditions are not specified in the examples, they are performed according to techniques or conditions described in the literature in the art or according to the product description. The reagents or instruments used are conventional products that can be obtained commercially without indicating the manufacturer.

### Example 1

### Combination of Ellagic Acid and Citric Acid

Different concentrations of citric acid (CA) and ellagic acid (EA) were added to 25 ml of deionized water, the supersaturation of the solution was controlled at S=2.4, and the growth process of the crystal was monitored. The inhibition percentages of the combination of ellagic acid/citric acid in three directions of crystal growth were obtained, as shown in Table 1.

TABLE 1
Ellagic acid/citric acid combination pairs concentrations
and the inhibition percentages of crystal growth achieved

| Inhibition | [001] | | [021] | | [121̅] | |
|---|---|---|---|---|---|---|
| percentage | CA/mM | EA/μM | CA/mM | EA/μM | CA/mM | EA/μM |
| 10% | 0.0108 | 0 | 0.0121 | 0 | 0.0135 | 0 |
| | 0 | 0.0105 | 0 | 0.0117 | 0 | 0.0164 |
| | 0.0009 | 0.0012 | 0.0015 | 0.0019 | 0.0023 | 0.0030 |
| 30% | 0.0369 | 0 | 0.0409 | 0 | 0.0458 | 0 |
| | 0 | 0.0386 | 0 | 0.0398 | 0 | 0.0562 |
| | 0.0034 | 0.0044 | 0.0051 | 0.0067 | 0.0080 | 0.0104 |
| 50% | 0.0710 | 0 | 0.0795 | 0 | 0.0890 | 0 |
| | 0 | 0.0761 | 0 | 0.0772 | 0 | 0.1100 |
| | 0.0066 | 0.0086 | 0.0101 | 0.0132 | 0.0154 | 0.0200 |
| 70% | 0.1254 | 0 | 0.1380 | 0 | 0.1550 | 0 |
| | 0 | 0.1322 | 0 | 0.1369 | 0 | 0.1884 |
| | 0.0115 | 0.0149 | 0.0175 | 0.0228 | 0.0268 | 0.0349 |

*CA refers to citric acid, and EA refers to ellagic acid.

According to the data in the table, when CA acts alone at a concentration of 0.0108 mM, the inhibition degree on the crystal growth in direction reaches 10%; when EA acts alone at a concentration of 0.0105 μM, the inhibition degree on the crystal growth in the direction reaches 10%; but when CA and EA are used in combination, the combination of 0.0009 mM CA and 0.0012 μM EA can achieve 10% growth inhibition on the COM crystal in the [001] direction. The combination of CA and EA increased the effectiveness of CA and EA by 12 times and 8.8 times, respectively.

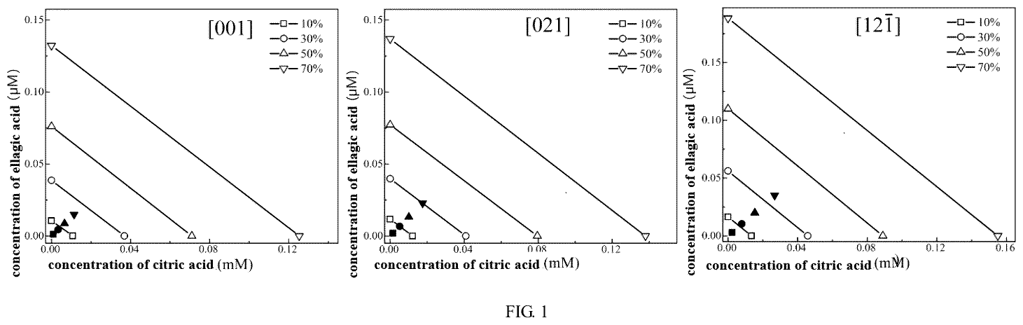

The effect of the combination of inhibitors was plotted in an isobologram, as shown in FIG. 1. The hollow symbols in the figure represent the concentration required for a single inhibitor to achieve a certain effect, and the same solid symbols represent the concentration required for a combination of the inhibitors to achieve the same effect. If the solid symbol falls to the lower left of the line between the hollow symbols, it indicates that the combination has a synergistic effect; if the solid symbol falls on the line, it indicates that the effect of the combination is additive; and if the solid symbol falls to the upper right of the line, it indicates that the effect of the combination is antagonistic.

FIG. 1 clearly shows that the combination of ellagic acid/citric acid exhibits a synergistic effect, and compared with the individual effects of the inhibitor molecules, the combination improves the performance of ellagic acid by nearly 8 times and the performance of citric acid by nearly 10 times, respectively.

### Example 2

### Combination of Gallic Acid and Citric Acid

Different concentrations of citric acid (CA) and gallic acid (GaA) were added to 25 ml of deionized water, and the growth process of the crystal was monitored. The inhibition percentages of the combination of gallic acid/citric acid in three directions of crystal growth were obtained, as shown in Table 2.

TABLE 2
Gallic acid/citric acid combination pairs concentrations
and the inhibition percentages of crystal growth achieved

| Inhibi- | | | | | | |
|---|---|---|---|---|---|---|
| tion | | | | | | |
| percent- | [001] | | [021] | | [121̅] | |
| age | CA/mM | GaA/μM | CA/mM | GaA/μM | CA/mM | GaA/μM |
| 10% | 0.0108 | 0 | 0.0121 | 0 | 0.0135 | 0 |
| | 0 | 0.4678 | 0 | 0.3743 | 0 | 0.4959 |
| | 0.0013 | 0.0311 | 0.0018 | 0.0444 | 0.0034 | 0.0844 |
| 30% | 0.0369 | 0 | 0.0409 | 0 | 0.0458 | 0 |
| | 0 | 1.5906 | 0 | 1.2912 | 0 | 1.6841 |
| | 0.0045 | 0.1110 | 0.0061 | 0.1510 | 0.0115 | 0.2842 |
| 50% | 0.0710 | 0 | 0.0795 | 0 | 0.0890 | 0 |
| | 0 | 3.0970 | 0 | 2.5075 | 0 | 3.2747 |
| | 0.0090 | 0.2176 | 0.0120 | 0.2975 | 0.0225 | 0.5551 |
| 70% | 0.1254 | 0 | 0.1380 | 0 | 0.1550 | 0 |
| | 0 | 5.3799 | 0 | 4.3601 | 0 | 5.6887 |
| | 0.0156 | 0.3864 | 0.0210 | 0.5196 | 0.0390 | 0.9637 |

According to the data in the table, when CA acts alone at a concentration of 0.0108 mM, the inhibition degree on the crystal growth in direction reaches 10%; when GaA acts alone at a concentration of 0.4678 μM, the inhibition degree on the crystal growth in the [001] direction reaches 10%; but when CA and GaA are used in combination, the combination of 0.0013 mM CA and 0.0311 μM GaA can achieve 10% growth inhibition on the COM crystal in the direction. The combination of CA and GaA increased the effectiveness of CA and GaA by 8.3 times and 15 times, respectively.

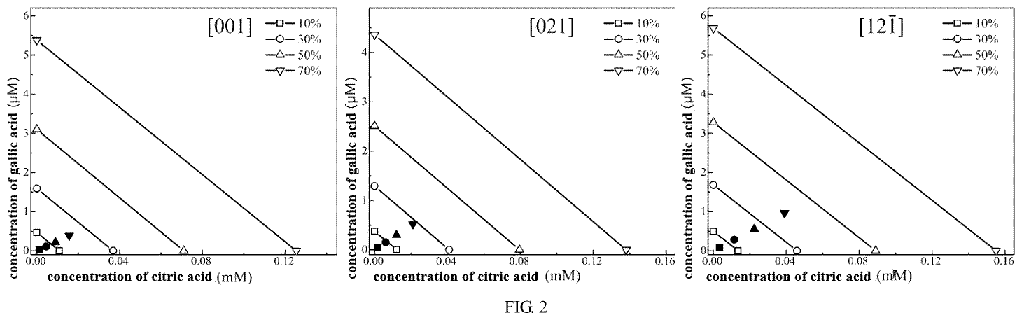

The effect of the combination of inhibitors was plotted in an isobologram, as shown in FIG. 2. As can be seen from the figure, the combination of gallic acid/citric acid exhibits a synergistic effect, and compared with the individual effects of the inhibitor molecules, the combination improves the performance of gallic acid by nearly 14 times and the performance of citric acid by nearly 7 times, respectively.

### Example 3

### Combination of Pyrogallol and Citric Acid

Different concentrations of citric acid (CA) and pyrogallol (PGA) were added to 25 ml of deionized water, and the growth process of the crystal was monitored. The inhibition percentages of the combination of pyrogallol/citric acid in three directions of crystal growth were obtained, as shown in Table 3.

TABLE 3
Pyrogallol/citric acid combination pairs concentrations
and the inhibition percentages of crystal growth achieved

| Inhibi- | | | | | | |
|---|---|---|---|---|---|---|
| tion | | | | | | |
| percent- | [001] | | [021] | | [121̅] | |
| age | CA/mM | PGA/mM | CA/mM | PGA/mM | CA/mM | PGA/mM |
| 10% | 0.0108 | 0 | 0.0121 | 0 | 0.0135 | 0 |
| | 0 | 0.0021 | 0 | 0.0018 | 0 | 0.0027 |
| | 0.0043 | 0.0007 | 0.0043 | 0.0007 | 0.0046 | 0.0007 |
| 30% | 0.0369 | 0 | 0.0409 | 0 | 0.0458 | 0 |
| | 0 | 0.0073 | 0 | 0.0062 | 0 | 0.0091 |
| | 0.0146 | 0.0023 | 0.0148 | 0.0023 | 0.0162 | 0.0025 |
| 50% | 0.0710 | 0 | 0.0795 | 0 | 0.0890 | 0 |
| | 0 | 0.0142 | 0 | 0.0121 | 0 | 0.0177 |
| | 0.0283 | 0.0044 | 0.0291 | 0.0045 | 0.0313 | 0.0049 |
| 70% | 0.1254 | 0 | 0.1380 | 0 | 0.1550 | 0 |
| | 0 | 0.0245 | 0 | 0.0210 | 0 | 0.0309 |
| | 0.0493 | 0.0077 | 0.0504 | 0.0079 | 0.0547 | 0.0085 |

According to the data in the table, when CA acts alone at a concentration of 0.0108 mM, the inhibition degree on the crystal growth in direction reaches 10%; when PGA acts alone at a concentration of 0.0021 mM, the inhibition degree on the crystal growth in the direction reaches 10%; but when CA and PGA are used in combination, the combination of 0.0043 mM CA and 0.0007 mM PGA can achieve 10% growth inhibition on the COM crystal in the [001] direction. The combination of CA and PGA increased the effectiveness of CA and PGA by 3 times and 2.5 times, respectively.

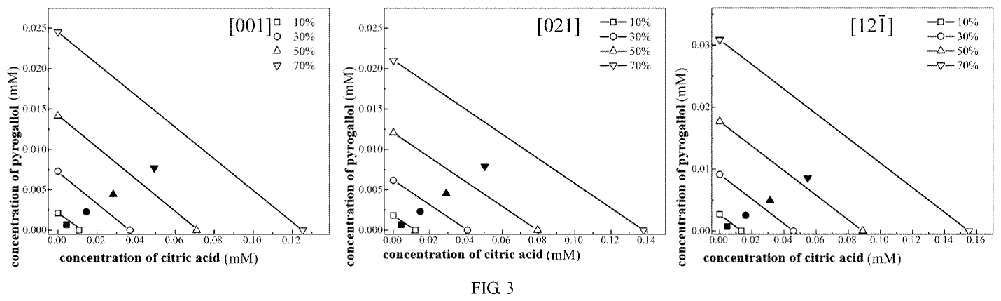

The effect of the combination of inhibitors was plotted in an isobologram, as shown in FIG. 3. As can be seen from the figure, the combination of pyrogallol/citric acid exhibits a synergistic effect, and compared with the individual effects of the inhibitor molecules, the combination improves the performance of pyrogallol by nearly 3 times and the performance of citric acid by nearly 2.5 times, respectively.

### Example 4

### Combination of Protocatechuic Acid and Citric Acid

Different concentrations of citric acid (CA) and protocatechuic acid (PrA) were added to 25 ml of deionized water, and the growth process of the crystal was monitored. The inhibition percentages of the combination of protocatechuic acid/citric acid in three directions of crystal growth were obtained, as shown in Table 4.

TABLE 4
Protocatechuic acid/citric acid combination pairs concentrations
and the inhibition percentages of crystal growth achieved

| Inhibi- | | | | | | |
|---|---|---|---|---|---|---|
| tion | | | | | | |
| percent- | [001] | | [021] | | [121̅] | |
| age | CA/mM | PrA/mM | CA/mM | PrA/mM | CA/mM | PrA/mM |
| 10% | 0.0108 | 0 | 0.0121 | 0 | 0.0135 | 0 |
| | 0 | 0.0255 | 0 | 0.0187 | 0 | 0.0307 |
| | 0.0006 | 0.0020 | 0.0009 | 0.0016 | 0.0010 | 0.0024 |
| 30% | 0.0369 | 0 | 0.0409 | 0 | 0.0458 | 0 |
| | 0 | 0.0982 | 0 | 0.0702 | 0 | 0.1174 |
| | 0.0022 | 0.0061 | 0.0032 | 0.0052 | 0.0040 | 0.0086 |
| 50% | 0.0710 | 0 | 0.0795 | 0 | 0.0890 | 0 |
| | 0 | 0.2640 | 0 | 0.1865 | 0 | 0.3159 |
| | 0.0048 | 0.0123 | 0.0073 | 0.0112 | 0.0112 | 0.0212 |

According to the data in the table, when CA acts alone at a concentration of 0.0108 mM, the inhibition degree on the crystal growth in direction reaches 10%; when PrA acts alone at a concentration of 0.0255 mM, the inhibition degree on the crystal growth in the direction reaches 10%; but when CA and PrA are used in combination, the combination of 0.0006 mM CA and 0.0020 mM PrA can achieve 10% growth inhibition on the COM crystal in the [001] direction. The combination of CA and PrA increased the effectiveness of CA and PrA by 12.8 times and 18 times, respectively.

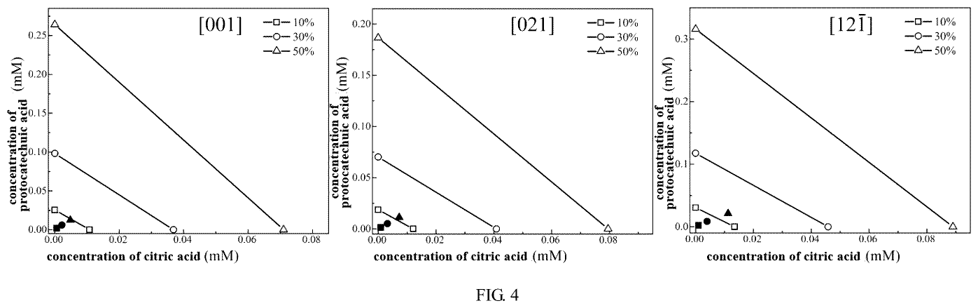

The effect of the combination of inhibitors was plotted in an isobologram, as shown in FIG. 4. As can be seen from the figure, the combination of protocatechuic acid/citric acid exhibits a synergistic effect, and compared with the individual effects of the inhibitor molecules, the combination improves the performance of protocatechuic acid by nearly 12 times and the performance of citric acid by nearly 17 times, respectively.

### Example 5

### Synergistic Inhibition of a Pharmaceutical Combination on CaOx Stone Formation

Studies have shown that reducing calcium oxalate crystallization, crystal-cell interactions, and cellular damage can prevent stone formation to some extent. On this basis, the present example further verified the combined effect of the pharmaceutical composition of citric acid (200 μM) and polyphenol molecules (20 μM GaA, 20 μM EA, 40 μM PGA, 200 UM PrA) in reducing cellular damage and crystal adhesion.

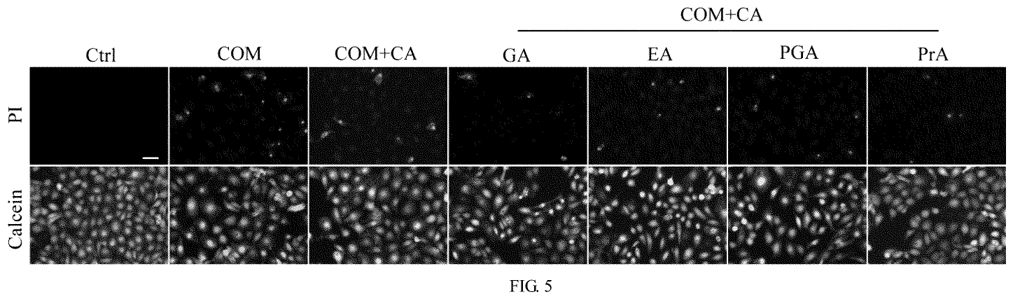

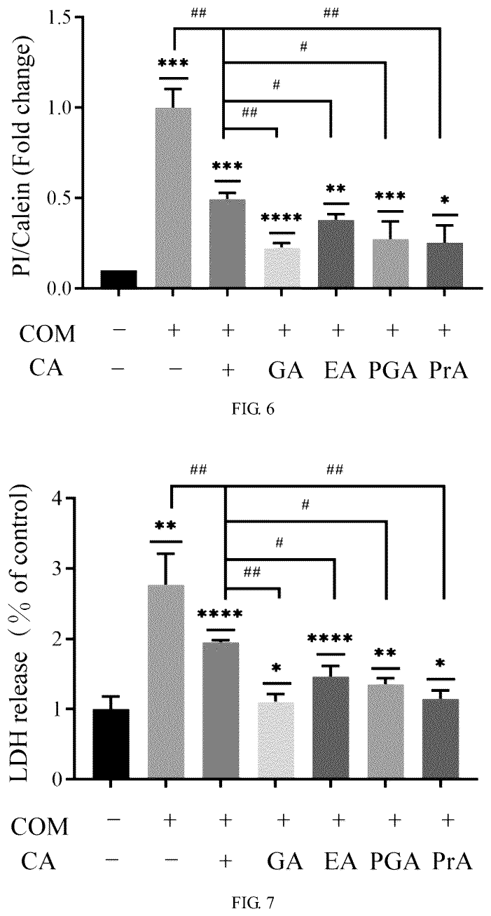

In order to explore the combined effects of the pharmaceutical composition on reducing crystal toxicity to cells, an in vitro model of direct exposure to COM crystals was selected in the present example to detect short-term cellular damage. In a culture medium environment with or without inhibitors, mature human proximal tubule cells (HK-2) were exposed to COM crystal suspension medium for the indicated times. Compared with HK-2 cells in the control group, the cells exposed to COM crystals after 24 hours showed decreased membrane integrity and reduced cellular lactonase activity (FIG. 5). However, in the presence of CA, the amount of cell death was reduced by 50% (FIG. 6). In addition, FIG. 6 also shows the synergistic effect of the pharmaceutical composition of the four polyphenol molecules and citric acid in improving cell activity, and the synergistic enhancement order is: GaA>PcA>PGA>EA.

Lactate dehydrogenase (LDH) is a cytoplasmic enzyme released into the extracellular space when the cell membrane ruptures, so the amount of LDH released can be used as a sign of cell membrane integrity. As shown in FIG. 7, the LDH release from cells directly exposed to COM crystals increased to 277.14% (+36.02%) compared with the control group, and decreased to 195.17% (+2.46%) in the presence of 200 μM CA. The combination of CA and GaA can reduce cellular LDH release to 110.87% (+8.67%), which further reveals the synergistic effect of the combination of CA and polyphenols in reducing crystal toxicity.

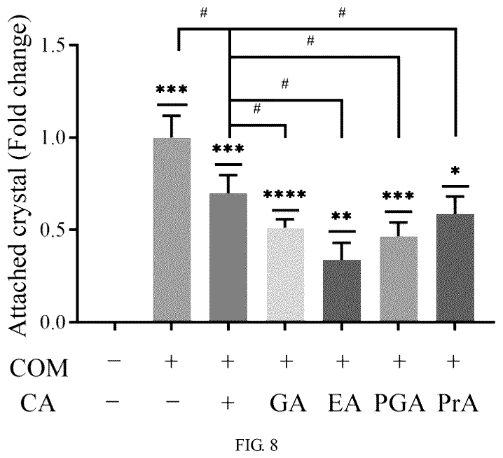

Crystal adhesion is not only a prerequisite for cell necrosis, but also the key to trigger crystal aggregation. Therefore, in this example, the combined inhibitory effect of the pharmaceutical combination on the adhesion of COM crystals to HK-2 cells in vitro was investigated by quantifying the amount of crystal adhesion (FIG. 8). The results showed that compared with the blank group without inhibitor, the inhibitory effect of 200 μM CA on COM crystal adhesion reached 30%. When used in combination with polyphenol molecules (GaA, EA, PGA, or PrA), the amount of crystal adhesion can be reduced by up to 50% by CA, and the combination of EA/CA is the most effective inhibitor combination for inhibiting crystal adhesion.

### Comparative Example 1

Comparative Example 1 differed from Example 1 merely in that ellagic acid was replaced with albumin, and the other preparation methods were the same as those in Example 1.

By comparing the experimental phenomena, it was found that the combination of albumin and citric acid actually reduced the inhibitory performance of citric acid.

### Comparative Example 2

Comparative Example 2 differed from Example 1 merely in that ellagic acid was replaced with o-hydroxybenzoic acid, and the other preparation methods were the same as those in Example 1.

By comparing the experimental phenomena, it was found that the combination of o-hydroxybenzoic acid and citric acid actually reduced the inhibitory performance of citric acid.

### Example 6

### Combination of Ellagic Acid and Hydroxycitric Acid

Different concentrations of hydroxycitric acid (HCA) and ellagic acid (EA) were added to 25 ml of deionized water, supersaturation of the solution was controlled at S=2.4, and the growth process of the crystal was monitored. The inhibition percentages of the combination of ellagic acid/hydroxycitric acid in three directions of crystal growth were obtained, as shown in Table 5.

TABLE 5
Ellagic acid/hydroxycitric acid combination pairs concentrations
and the inhibition percentages of crystal growth achieved

| Inhibi- | | | | | | |
|---|---|---|---|---|---|---|
| tion | | | | | | |
| percent- | [001] | | [021] | | [121̅] | |
| age | HCA/mM | EA/μM | HCA/mM | EA/μM | HCA/mM | EA/μM |
| 10% | 0.0088 | 0 | 0.0096 | 0 | 0.0110 | 0 |
| | 0 | 0.0084 | 0 | 0.0094 | 0 | 0.0131 |
| | 0.0007 | 0.0009 | 0.0012 | 0.0016 | 0.0016 | 0.0024 |
| 30% | 0.0281 | 0 | 0.0320 | 0 | 0.0367 | 0 |
| | 0 | 0.0309 | 0 | 0.0320 | 0 | 0.0449 |
| | 0.0027 | 0.0035 | 0.0040 | 0.0055 | 0.0072 | 0.0080 |
| 50% | 0.0561 | 0 | 0.0610 | 0 | 0.0720 | 0 |
| | 0 | 0.0609 | 0 | 0.0560 | 0 | 0.0888 |
| | 0.0049 | 0.0073 | 0.0081 | 0.0104 | 0.0121 | 0.0160 |
| 70% | 0.1003 | 0 | 0.1104 | 0 | 0.1240 | 0 |
| | 0 | 0.1041 | 0 | 0.1095 | 0 | 0.1500 |
| | 0.0096 | 0.0120 | 0.0144 | 0.01824 | 0.0214 | 0.0280 |

*HCA refers to hydroxycitric acid, and EA refers to ellagic acid.

According to the data in the table, when HCA acts alone at a concentration of 0.0088 mM, the inhibition degree on the crystal growth in direction reaches 10%; when EA acts alone at a concentration of 0.0084 μM, the inhibition degree on the crystal growth in the direction reaches 10%; but when HCA and EA are used in combination, the combination of 0.0007 mM HCA and 0.0009 UM EA can achieve a 10% growth inhibition on the COM crystal in the [001] direction. The combination of HCA and EA increased the effectiveness of HCA and EA by 12.6 times and 9.3 times, respectively.

### Example 7

### Combination of Gallic Acid and Hydroxycitric Acid

Different concentrations of hydroxycitric acid (HCA) and gallic acid (GaA) were added to 25 ml of deionized water, and the growth process of the crystal was monitored. The inhibition percentages of the combination of gallic acid/hydroxycitric acid in three directions of crystal growth were obtained, as shown in Table 6.

TABLE 6
Gallic acid/hydroxycitric acid combination pairs concentrations
and the inhibition percentages of crystal growth achieved

| Inhibition | [001] | | [021] | | [121̅] | |
|---|---|---|---|---|---|---|
| percentage | HCA/mM | GaA/μM | HCA/mM | GaA/μM | HCA/mM | GaA/μM |
| 10% | 0.0088 | 0 | 0.0096 | 0 | 0.0110 | 0 |
| | 0 | 0.3716 | 0 | 0.2994 | 0 | 0.3967 |
| | 0.0010 | 0.0250 | 0.0014 | 0.0350 | 0.0026 | 0.0675 |
| 30% | 0.0281 | 0 | 0.0320 | 0 | 0.0367 | 0 |
| | 0 | 1.2801 | 0 | 1.0221 | 0 | 1.3473 |
| | 0.0040 | 0.0818 | 0.0046 | 0.1200 | 0.0090 | 0.2274 |
| 50% | 0.0561 | 0 | 0.0610 | 0 | 0.0720 | 0 |
| | 0 | 2.4010 | 0 | 1.8001 | 0 | 2.6198 |
| | 0.0071 | 0.1656 | 0.0096 | 0.2400 | 0.0161 | 0.4411 |
| 70% | 0.1003 | 0 | 0.1104 | 0 | 0.1240 | 0 |
| | 0 | 4.3049 | 0 | 3.4880 | 0 | 4.5510 |
| | 0.0125 | 0.3091 | 0.0161 | 0.4157 | 0.0320 | 0.8274 |

According to the data in the table, when HCA acts alone at a concentration of 0.0088 mM, the inhibition degree on the crystal growth in direction reaches 10%; when GaA acts alone at a concentration of 0.3716 UM, the inhibition degree on the crystal growth in the [001] direction reaches 10%; but when HCA and GaA are used in combination, the combination of 0.0010 mM HCA and 0.0250 UM GaA can achieve 10% growth inhibition on the COM crystal in the direction. The combination of HCA and GaA increased the effectiveness of HCA and GaA by 8.8 times and 14.8 times, respectively.

### Example 8

### Combination of Ellagic Acid and Potassium Citrate

Different concentrations of potassium citrate (KCA) and ellagic acid (EA) were added to 25 ml of deionized water, the supersaturation of the solution was controlled at S=2.4, and the growth process of the crystal was monitored. The inhibition percentages of the combination of ellagic acid/potassium citrate in three directions of crystal growth were obtained, as shown in Table 7.

TABLE 7
Ellagic acid/potassium citrate combination pairs concentrations
and the inhibition percentages of crystal growth achieved

| Inhibi- | | | | | | |
|---|---|---|---|---|---|---|
| tion | | | | | | |
| percent- | [001] | | [021] | | [121̅] | |
| age | KCA/mM | EA/μM | KCA/mM | EA/μM | KCA/mM | EA/μM |
| 10% | 0.0106 | 0 | 0.0123 | 0 | 0.0135 | 0 |
| | 0 | 0.0104 | 0 | 0.0116 | 0 | 0.0165 |
| | 0.0008 | 0.0012 | 0.0016 | 0.0017 | 0.0024 | 0.0033 |
| 30% | 0.0369 | 0 | 0.0401 | 0 | 0.0451 | 0 |
| | 0 | 0.0385 | 0 | 0.0399 | 0 | 0.0561 |
| | 0.0035 | 0.0043 | 0.0053 | 0.0065 | 0.0077 | 0.0100 |
| 50% | 0.0709 | 0 | 0.0794 | 0 | 0.0881 | 0 |
| | 0 | 0.0763 | 0 | 0.0771 | 0 | 0.1099 |
| | 0.0068 | 0.0089 | 0.0100 | 0.0132 | 0.0153 | 0.0200 |
| 70% | 0.1253 | 0 | 0.1369 | 0 | 0.1547 | 0 |
| | 0 | 0.1312 | 0 | 0.1358 | 0 | 0.1879 |
| | 0.0114 | 0.0147 | 0.0171 | 0.0221 | 0.0261 | 0.0341 |

*KCA refers to potassium citrate, and EA refers to ellagic acid.

According to the data in the table, when KCA acts alone at a concentration of 0.0106 mM, the inhibition degree on the crystal growth in direction reaches 10%; when EA acts alone at a concentration of 0.0104 UM, the inhibition degree on the crystal growth in the [001] direction reaches 10%; but when KCA and EA are used in combination, the combination of 0.0008 mM KCA and 0.0012 μM EA can achieve 10% growth inhibition on the COM crystal in the direction. The combination of KCA and EA increased the effectiveness of KCA and EA by 13 times and 8.8 times, respectively.

### Example 9

### Combination of Gallic Acid and Potassium Citrate

Different concentrations of potassium citrate (KCA) and gallic acid (GaA) were added to 25 ml of deionized water, and the growth process of the crystal was monitored. The inhibition percentages of the combination of gallic acid/potassium citrate in three directions of crystal growth were obtained, as shown in Table 8.

TABLE 8
Gallic acid/potassium citrate combination pairs concentrations
and the inhibition percentages of crystal growth achieved

| Inhibition | [001] | | [021] | | [121̅] | |
|---|---|---|---|---|---|---|
| percentage | KCA/mM | GaA/μM | KCA/mM | GaA/μM | KCA/mM | GaA/μM |
| 10% | 0.0106 | 0 | 0.0123 | 0 | 0.0135 | 0 |
| | 0 | 0.4677 | 0 | 0.3744 | 0 | 0.4959 |
| | 0.0012 | 0.0312 | 0.0019 | 0.0445 | 0.0033 | 0.0843 |
| 30% | 0.0369 | 0 | 0.0401 | 0 | 0.0451 | 0 |
| | 0 | 1.5900 | 0 | 1.2910 | 0 | 1.6842 |
| | 0.0044 | 0.1108 | 0.0060 | 0.1511 | 0.0116 | 0.2843 |
| 50% | 0.0709 | 0 | 0.0794 | 0 | 0.0881 | 0 |
| | 0 | 3.0965 | 0 | 2.5072 | 0 | 3.2745 |
| | 0.0091 | 0.2173 | 0.0119 | 0.2974 | 0.0223 | 0.5550 |
| 70% | 0.1253 | 0 | 0.1369 | 0 | 0.1547 | 0 |
| | 0 | 5.3797 | 0 | 4.3600 | 0 | 5.6881 |
| | 0.0155 | 0.3863 | 0.0209 | 0.5197 | 0.0388 | 0.9634 |

According to the data in the table, when KCA acts alone at a concentration of 0.0106 mM, the inhibition degree on the crystal growth in direction reaches 10%; when GaA acts alone at a concentration of 0.4677 μM, the inhibition degree on the crystal growth in the [001] direction reaches 10%; but when KCA and GaA are used in combination, the combination of 0.0012 mM KCA and 0.0312 μM GaA can achieve 10% growth inhibition on the COM crystal in the direction. The combination of KCA and GaA increased the effectiveness of KCA and GaA by 8.8 times and 15 times, respectively. It can be seen that the effects of potassium citrate and citric acid are almost the same, and the effect of citrate mainly depends on the citrate ion.

In the specification, the description of the reference terms such as “one embodiment”, “some embodiments”, “example”, “specific example”, or “some examples” means that the specific features, structures, materials, or characteristics described with reference to the embodiment or example are included in at least an embodiment or example of the present disclosure. In this specification, exemplary descriptions of the foregoing terms do not necessarily refer to the same embodiment or example. Moreover, the specific features, structures, materials, or characteristics described may be combined in any one or more embodiments or examples in a suitable manner. Furthermore, those skilled in the art may combine different embodiments or examples and features of different embodiments or examples described in this specification, unless they are contradictory to each other.

The applicant declares that the above-mentioned descriptions are only specific embodiments of the present disclosure, but the protection scope of the present disclosure is not limited thereto. Those skilled in the art should understand that any changes or substitutions that can be easily thought of by those skilled in the art within the technical scope disclosed by the present disclosure are within the protection scope and disclosure scope of the present disclosure.

1. A pharmaceutical composition, comprising:
component A comprising polyphenol having 6 to 14 carbon atoms and/or a substance that is metabolizable metabolized into polyphenol having 6 to 14 carbon atoms in human body; and
component B comprising any one of citric acid, citrate, citric acid derivative, or citric acid derivative, or a combination of at least two thereof.

2. The pharmaceutical composition according to claim 1, wherein the polyphenol having 6 to 14 carbon atoms comprises any one of ellagic acid, gallic acid, pyrogallol, or protocatechuic acid, or a combination of at least two thereof.

3. The pharmaceutical composition according to claim 1, wherein the substance that is metabolizable into the polyphenol having 6 to 14 carbon atoms in the human body comprises any one of tannic acid, punicalagin, galloylglucose, or esterified catechin, or a combination of at least two thereof.

4. The pharmaceutical composition according to claim 1, wherein the citrate comprises any one of potassium citrate, sodium citrate, or magnesium citrate, or a combination of at least two thereof.

5. The pharmaceutical composition according to claim 1, wherein the citric acid derivative comprises hydroxycitric acid, an ester compound derived from citric acid, or a lactone compound derived from citric acid.

6. The pharmaceutical composition according to claim 1, wherein the citric acid derivative salt comprises any one of potassium hydroxycitrate and/or sodium hydroxycitrate, or magnesium hydroxycitrate, or a combination of at least two thereof.

7. The pharmaceutical composition according to claim 1, wherein a molar ratio of the component A to the component B in the pharmaceutical composition is (0.0013 to 3.33): 1.

8. The pharmaceutical composition according to claim 1, wherein:
a combined effecting concentration ratio of the component A and the component B is set based on effect mechanisms of the respective components; wherein:
when the effect mechanisms of the component A and the component B are the same, the molar ratio of the component A to the component B is (0.0013 to 0.163): 1;
when the effect mechanisms of the component A and the component B are similar, the molar ratio of the component A to the component B is (0.0023 to 0.025): 1; or
when the effect mechanisms of the component A and the component B are different, the molar ratio of the component A to the component B is (1.77 to 3.33): 1.

9. The pharmaceutical composition according to claim 7, wherein:
the pharmaceutical composition comprises ellagic acid with a molar concentration ranging from 0 μM to 2 μM and the component B with a molar concentration ranging from 0 μM to 0.5 mM; and
an addition amount of the ellagic acid and an addition amount of the component B are not zero.

10. The pharmaceutical composition according to claim 7, wherein:
the pharmaceutical composition comprises gallic acid with a molar concentration ranging from 0 μM to 25 μM and the component B with a molar concentration ranging from 0 mM to 0.5 mM; and
an addition amount of the gallic acid and an addition amount of the component B are not zero.

11. The pharmaceutical composition according to claim 7, wherein:
the pharmaceutical composition comprises pyrogallol with a molar concentration ranging from 0 mM to 15 mM and the component B with a molar concentration ranging from 0 mM to 0.5 mM; and
an addition amount of the pyrogallol and an addition amount of the component B are not zero.

12. The pharmaceutical composition according to claim 7, wherein:
the pharmaceutical composition comprises protocatechuic acid with a molar concentration ranging from 0 mM to 0.55 mM and the component B with a molar concentration ranging from 0 mM to 0.5 mM; and
an addition amount of the protocatechuic acid and an addition amount of the component B are not zero.

13. The pharmaceutical composition according to claim 1, further comprising a pharmaceutically acceptable carrier or excipient.

14. The pharmaceutical composition according to claim 13, wherein the carrier comprises any one of liposomes, micelles, dendritic macromolecules, microspheres, or microcapsules, or a combination of at least two thereof.

15. The pharmaceutical composition according to claim 13, wherein the excipient comprises any one of a wetting agent, a binder, or a disintegrant, or a combination of at least two thereof.

16. The pharmaceutical composition according to claim 1, wherein the pharmaceutical composition is in a formulation of compound preparation.

17. The pharmaceutical composition according to claim 1, wherein the pharmaceutical composition is in a formulation of a separate preparation of the component A and a separate preparation of the component B.

18. The pharmaceutical composition according to claim 17, wherein an administration of the separate preparation comprises simultaneous administration, separate administration, or sequential administration.

19. (canceled)

20. The pharmaceutical composition according to claim 1, wherein the dosage form comprises any one of tablets, granules, powders for solution, pulveres, capsules, or injections.

21. (canceled)

22. (canceled)

23. (canceled)

24. A method for preventing and/or treating kidney stones, comprising:
administering an effective amount of the pharmaceutical composition according to claim 1 to a subject suffering from or suspected of suffering from kidney stones.
